(19) United States
(12) Patent Application Publication
(10) Pub. No.: US 2023/0240402 A1
(43) Pub. Date: Aug. 3, 2023

(54) Magnetic Lash Band System

(71) Applicant: Glamnetic LLC, Los Angeles, CA (US)

(72) Inventor: Ann McFerran, Miami, FL (US)

(21) Appl. No.: 17/589,150

(22) Filed: Jan. 31, 2022

(51) Int. Cl. A41G 5/02; A61K 8/29; A61K 8/19; A61K 8/34; A61K 8/81; A61K 8/73; A61Q 1/10

(52) U.S. Cl. CPC A41G 5/02; A61K 8/29; A61K 8/19; A61K 8/345; A61K 8/8147; A61K 8/73; A61Q 1/10; A61K 2800/47

(57) ABSTRACT

A false eyelash band is disclosed, including a substrate strip having a first surface and an opposing second surface. The substrate strip formed by consolidating a plurality of fibers, each of the plurality of fibers impregnated with magnetic element particles from a magnetic coating composition. The first surface configured to secure a plurality of artificial hairs along the length of the substrate strip, and the second surface comprising at least a magnetic coating layer comprising a magnetic substance and configured to be attached to a user.

### BACKGROUND

The present invention relates generally to false eyelash assemblies and, in particular but not exclusively, to magnetic lash bands to which false lashes are secured by non-adhesive means. False eyelashes are a type of cosmetic product that is used to enhance the physical characteristics of the wearer's natural eyelashes. For example, false eyelashes can be used to enhance the length, curl, fullness, and thickness of natural eyelashes.

False eyelash assemblies may consist of several individual false eyelashes, which are attached to a lash band—a narrow, elongated substrate—in various configurations. The lash band, and the false eyelashes supported thereon, can then be temporarily affixed to a wearer's eyelid. In the past, false eyelashes were temporarily affixed to a wearer's eyelid using an adhesive substance that was applied to the eyelid of the wearer, the lash band, or both. However, the use of adhesive substances to affix false eyelashes to a wearer's eyelid proved difficult, unpleasant, and often injurious to the wearer. The use of an adhesive substance to apply a false eyelash assembly to a wearer's eyelid makes it more difficult for a wearer to re-align or re-orient the assembly once it has been placed on a wearer's eyelid, particularly when the adhesive substance has had sufficient time to set. As such, a wearer is afforded very little room for error when applying a false eyelash assembly using an adhesive substance. Further, the use of adhesive substances can often irritate the skin and eyes of the wearer, either due to the adhesive substance itself, or resulting from the removal of a false eyelash assembly that has been adhered to a wearer's eyelid using an adhesive substance.

More recently, however, false eyelash assemblies were developed that were designed to be temporarily affixed to a wearer's eyelids using non-adhesive means, such as magnetic force. These false eyelash assemblies include a lash band that is provided with one or more magnetic elements. As such, false eyelash assemblies can be temporarily adhered to a wearer's eyelids without the use of an adhesive. However, false eyelash assemblies employing magnetic elements pose their own set of disadvantages. Currently, one or more magnets are secured to a skin-facing surface of a lash band, which reduces the flexibility of the lash band and the magnets protruding from the surface of the lash band prevents the lash band from lying flat against a wearer's eyelid. Besides the various aesthetic disadvantages posed by magnetically-attached false eyelash assemblies, such assemblies also pose a physical risk to wearers. The magnets employed by these false eyelash assemblies are capable of scratching and injuring a wearer's eyelid.

As more and more people seek to augment their natural eyelashes, false eyelash assemblies have become a staple cosmetic product, but the current use of adhesives and magnets have proven to make their daily use more difficult and, sometimes, even injurious to the health of its users. For example, current lashes may be uncomfortable to the eyes and sometimes cause irritation due to scratching the eyelids. Further, current false lashes, particularly those utilizing individual magnetic elements, are heavy and fail to blend in with a wearer's natural eyelashes. Therefore, there is a need for a false eyelash band that reliably secures artificial eyelash hairs to a wearer without the use of adhesives or notably visible magnets, which is lighter weight, will lessen or eliminate eyelid irritation, and will better blend in with a user's natural lashes.

### SUMMARY

The present invention discloses a false eyelash band and a false eyelash assembly to overcome the physical and aesthetic issues related to current false eyelash assemblies. These issues are solved, including through providing a false eyelash band comprising a substrate strip having a first surface and an opposing second surface. The first surface configured to secure a plurality of artificial hairs along the length of the substrate strip and the second surface configured to attach to a wearer's eyelid. The substrate strip is formed by consolidating a plurality of fibers which have been coated with a first magnetic coating composition, which includes magnetic element particles. The magnetic particles of the first magnetic coat composition will marry with magnetic particles applied to a user's eyelid in the form of a magnetic eyeliner. A second magnetic coating composition including a magnetic material, may also be applied on the second surface of the substrate strip to provide an additional attachment surface to a wearer's eyelid by marrying with magnetic particles applied to a user's eyelid in the form of a magnetic eyeliner to cause a stronger hold.

The false eyelash band of the present invention comprises a substrate strip having a first surface and an opposing second surface, the substrate strip formed by a consolidated plurality of fibers, each of the plurality of fibers impregnated with magnetic element particles from a magnetic coating composition, the first surface is configured to secure a plurality of artificial hairs along a length of the substrate strip, the second surface is configured to be attached to a user.

The false eyelash band of the present invention may further comprise a magnetic coating layer comprising a magnetic substance coated on the second surface of the substrate strip.

The present invention is also directed to a false eyelash assembly, comprising a substrate strip having a first surface and an opposing second surface, the substrate strip formed by a consolidated plurality of fibers, each of the plurality of fibers impregnated with magnetic element particles; and a magnetic coating layer including a magnetic substance coated on the second surface; and a plurality of artificial hairs spaced apart and secured along the length of the first surface.

### BRIEF DESCRIPTION OF THE SEVERAL VIEWS OF THE DRAWINGS

The accompanying drawings, which are incorporated in and constitute a part of this specification, illustrate one or more implementations described herein and, together with the description, explain these implementations. The drawings are not intended to be drawn to scale, and certain features and certain views of the figures may be shown exaggerated, to scale or in schematic in the interest of clarity and conciseness. Not every component may be labeled in every drawing. Like reference numerals in the figures may represent and refer to the same or similar element or function. In the drawings:

FIG. 1 is a perspective view of a false eyelash band in accordance with the present disclosure.

FIG. 2 is a cross-sectional view of one of a plurality of fibers forming a substrate strip in accordance with the present disclosure

FIG. 3 is a cross-sectional view of the substrate strip illustrated in FIG. 1.

FIG. 4 is a perspective view of a substrate strip having a plurality of artificial hairs attached to a surface thereof in accordance with the present disclosure.

### DETAILED DESCRIPTION

Before explaining at least one embodiment of the inventive concept disclosed herein in detail, it is to be understood that the inventive concept is not limited in its application to the details of construction and the arrangement of the components or steps or methodologies set forth in the following description or illustrated in the drawings. The inventive concept disclosed herein is capable of other embodiments or of being practiced or carried out in various ways. Also, it is to be understood that the phraseology and terminology employed herein is for the purpose of description and should not be regarded as limiting in any way. No element, act, or instruction used in the present application should be construed as critical or essential to the invention unless explicitly described as such.

In the following detailed description of embodiments of the inventive concept, numerous specific details are set forth in order to provide a more thorough understanding of the inventive concept. It will be apparent to one of ordinary skill in the art, however, that the inventive concept within the disclosure may be practiced without these specific details. In other instances, well-known features have not been described in detail to avoid unnecessarily complicating the instant disclosure.

As used herein, the terms “comprises”, “comprising”, “includes”, “including”, “has”, “having”, or any other variation thereof, are intended to be non-exclusive inclusions. For example, a process, method, article, or apparatus that comprises a set of elements is not limited to only those elements but may include other elements not expressly listed or even inherent to such process, method, article, or apparatus.

Further, unless expressly stated to the contrary, “or” refers to an inclusive or and not to an exclusive or. For example, a condition A or B is satisfied by anyone of the following: A is true (or present) and B is false (or not present), A is false (or not present) and B is true (or present), and both A and B are true (or present).

In addition, the use of the “a” or “an” are employed to describe elements and components of the embodiments herein. This is done merely for convenience and to give a general sense of the inventive concept. This description should be read to include one or more, and the singular also includes the plural unless it is obvious that it is meant otherwise.

Further, use of the term “plurality” is meant to convey “more than one” unless expressly stated to the contrary.

The use of ordinal number terminology (i.e., “first”, “second”, “third”, “fourth”, etc.) is solely for the purpose of differentiating between two or more items and, unless explicitly stated otherwise, is not meant to imply any sequence or order of importance to one item over another.

The use of the term “at least one” or “one or more” will be understood to include one as well as any quantity more than one. In addition, the use of the phrase “at least one of X, Y, and Z” will be understood to include X alone, Y alone, and Z alone, as well as any combination of X, Y, and Z.

Finally, as used herein any reference to “one embodiment” or “an embodiment” means that a particular element, feature, structure, or characteristic described in connection with the embodiment is included in at least one embodiment. The appearance of the phrase “in one embodiment” in various places in the specification are not necessarily all referring to the same embodiment.

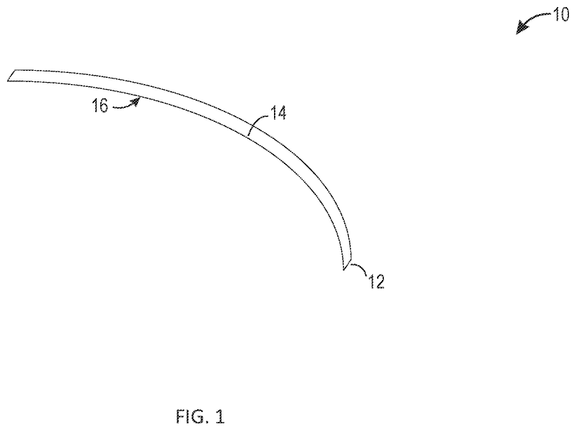

Referring now to FIG. 1, shown therein is a perspective view of an artificial eyelash band 10 in accordance with the present disclosure. Artificial eyelash band 10 comprises a substrate strip 12 having a first surface 14 and an opposing second surface 16. The first surface 14 of the substrate strip 12 may be configured to secure a plurality of artificial hairs along the length of the substrate strip 12, as discussed in further detail below. The second surface 16 of the substrate strip 12 may be configured to be attached to a user, as discussed in further detail below. The substrate strip 12 may be of any length including, for example, a length that substantially covers at least a portion of a wearer's eyelid. The substrate strip 12 may also be any shape including, for example, a shape that substantially matches the contours of a wearer's eyelid. The substrate strip 12 may be sufficiently flexible such that a wearer may readily manipulate the degree of curvature of the substrate strip 12 to match the contours of a particular wearer's eyelid. The substrate strip 12 may be formed by a consolidated plurality of fibers 18. The substrate strip 12 may be formed of natural or synthetic fibers such as, for example, polyamide, polyester, or cotton filaments. In one embodiment, the substrate strip 12 is formed by a consolidated plurality of black cotton fibers. Cotton is a widely used fiber type that exhibits, among other things, high absorbency. Any fibers suitable to be infused, impregnated, or coated with a film, paste, or other compound may be used to form the substrate strip 12.

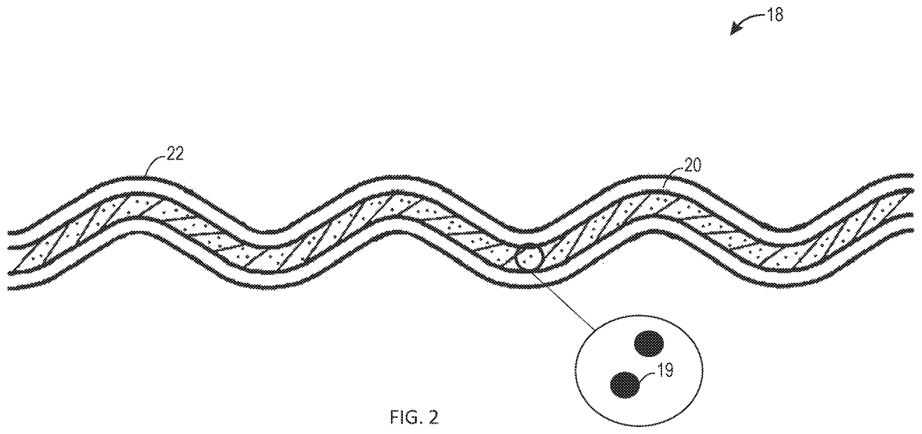

Referring now to FIG. 2, shown therein is a cross-sectional view of one of the plurality of fibers 18 forming the substrate strip 12. Each of the plurality of fibers 18 may have a cross-section of any suitable geometry, including, but not limited to, circular and oval. Each of the plurality of fibers 18 may be impregnated with magnetic material particles 19 from a magnetic coating composition 20. The magnetic coating composition 20 may be coated on a circumferential outer surface 22 of each of the plurality of fibers 18 along the length of each of the plurality of fibers 18.

The magnetic coating composition 20 includes magnetic material particles 19 such as for example, black iron oxide particles. The application of the magnetic coating composition 20 on each of the plurality of fibers 18 functionalizes the plurality of fibers 18, imparting magnetic properties to the plurality of fibers 18, and ultimately, the whole of the substrate strip 12 once the plurality of fibers 18 are consolidated to form the substrate strip 12. In one embodiment, the magnetic material may be a magnetic material, such as black iron oxide.

The magnetic coating composition 20 may also include, for example, various solvents, emollients, rheology modifiers, binders, thickening agents, and other substances known in the art for forming cosmetic products. In one embodiment the magnetic coating composition 20 may include, for example but not by way of limitation, (1) isododecane, (2) hydrogenated C6-20 polyolefin, (3) black iron oxide, (4) dimethicone, (5) cera microcristallina, (6) silica, (7) vinyl dimethicone/methicone silsesquioxane crosspolymer, (8) ceresin, (9) polyethylene, and (10 phenoxyethanol.

In that embodiment, the magnetic coating composition 20 may include isododecane in an amount of about 29.5 wt % to about 30 wt % hydrogenated C6-20 polyolefin in an amount of about 26.5 wt % to about 27 wt %; black iron oxide in an amount of about 26 wt % to about 26.5 wt %; dimethicone in an amount of about 4.5 wt % to about 5 wt %; cera microcristallina in an amount of about 2.5 wt % to about 3 wt %; silica in an amount of about 2.5 wt % to about 3 wt %; vinyl dimethicone/methicone silsesquioxane crosspolymer in an amount of about 1.5 wt % to about 2 wt %; ceresin in an amount of about 1.5 wt % to about 2 wt %; polyethylene in an amount of about 0.5 wt % to about 1 wt %; and phenoxyethanol in an amount of about 0.3 wt % to about 0.5 wt %.

In one embodiment, the magnetic coating composition 20 may be prepared by combining a magnetic element material with a solvent and other active materials to form a solution. Each of the plurality of fibers 18 may then be treated with the prepared magnetic solution such that the magnetic material particles 19 such as, for example, black iron oxide particles, are substantially uniformly dispersed within each of the plurality of fibers 18. The application of the prepared magnetic solution may be carried out by any suitable method known in the art such as, for example, the pad-dry-cure method. In one embodiment, the plurality of fibers 18 may be saturated with the solution for approximately 15 minutes to about 30 minutes. Or, the plurality of fibers 18 may be saturated with the solution until the plurality of fibers 18 reach a pick-up of about 96% to 98%. Thereafter, each of the plurality of fibers 18 may be heated to a temperature of about 60° C. to about 70° C. for approximately 30 minutes to about 40 minutes. In one embodiment, each of the plurality of fibers may be heated to a temperature of about 30° C. to about 40° C. for approximately 5 to 6 minutes.

The magnetic coating composition 20 may comprise (i) water, (ii) a solvent, (iii) a magnetic substance, (iv) an emulsifier, (v) a film forming agent, (vi) a surfactant, and (vii) a thickener. In one embodiment the magnetic coating layer 24 may include, for example but not by way of limitation, (1) water, (2) propylene glycol, (3) black iron oxide, (4) titanium dioxide, (5) styrene acrylate copolymer, (6) ethylhexylglycerin, (7) xanthan gum.

In one embodiment, the magnetic coating composition 20 may include water in an amount of about 45 wt % to about 46 wt % or about 46 wt %; propylene glycol in an amount of about 5 wt % to about 6 wt % or about 6 wt %; black iron oxide in an amount of about 9 wt % to about 10 wt % or about 10 wt %; titanium dioxide in an amount of about 22 wt % to about 23 wt % or about 23 wt %; styrene acrylate copolymer in an amount of about 14 wt % to about 14.6 wt % or about 14.6 wt %; ethylhexylglycerin in an amount of about 0.1 wt % to about 0.3 wt % or about 0.3 wt %; and xanthan gum in an amount of about 0.05 wt % to about 0.1 wt % or about 0.1 wt %.

Once each of the plurality of fibers 18 is impregnated with the magnetic element particles 19 of the magnetic coating composition 20, each of the plurality of fibers 20 may be consolidated by knitting or weaving to form the substrate strip 12. In one embodiment, each of the plurality of fibers 20 may be consolidated using a machine to weave each of the plurality of fibers 20 to form the substrate strip 12. Any machine known in the art for weaving individual fibers may be employed to weave each of the plurality of fibers 20 to form the substrate strip 12.

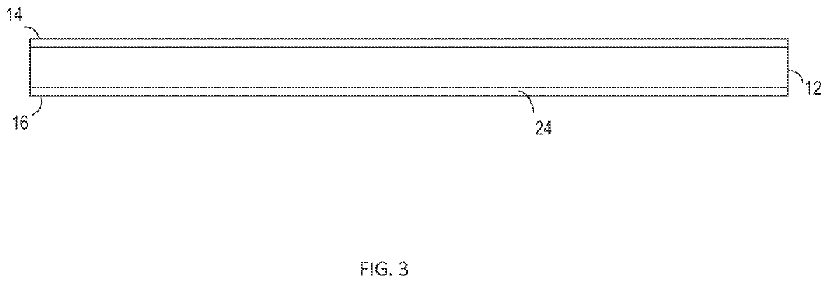

Referring now to FIG. 3, shown therein cross-sectional view of the substrate strip 12. The substrate strip 12 may be coated with a magnetic coating layer 24 on the second surface 16 as shown in FIG. 3.

The magnetic coating layer 24 may be coated on the second surface 16 in thin layers ranging from about 0.5 mm to 0.8 mm. The magnetic coating layer 24 may have a greater viscosity than the first coating layer 20. In one embodiment, the magnetic coating layer 24 may be prepared as a paste. The magnetic coating layer 24 may impart additional magnetic properties to the substrate strip 12 so as to provide for a stronger hold when applied to a wearer's eyelid.

Similar to the magnetic coating composition 20, the magnetic coating layer 24 may also comprise (i) water, (ii) a solvent, (iii) a magnetic substance, (iv) an emulsifier, (v) a film forming agent, (vi) a surfactant, and (vii) a thickener. In one embodiment the magnetic coating layer 24 may include, for example but not by way of limitation, (1) water, (2) propylene glycol, (3) black iron oxide, (4) titanium dioxide, (5) styrene acrylate copolymer, (6) ethylhexylglycerin, (7) xanthan gum. In one embodiment, the magnetic coating layer 20 may be prepared by combining water with the other active material to form a paste, which is thereafter coated on the second surface 16, and then subsequently dried. The magnetic coating layer 24 may be dried at ambient temperature or at a temperature of about 60° C. to about 70° C.

In one embodiment, the magnetic coating layer 24 may include water in an amount of about 45 wt % to about 46 wt % or about 46 wt %; propylene glycol in an amount of about 5 wt % to about 6 wt % or about 6 wt %; black iron oxide in an amount of about 9 wt % to about 10 wt % or about 10 wt %; titanium dioxide in an amount of about 22 wt % to about 23 wt % or about 23 wt %; styrene acrylate copolymer in an amount of about 14 wt % to about 14.6 wt % or about 14.6 wt %; ethylhexylglycerin in an amount of about 0.1 wt % to about 0.3 wt % or about 0.3 wt %; and xanthan gum in an amount of about 0.05 wt % to about 0.1 wt % or about 0.1 wt %.

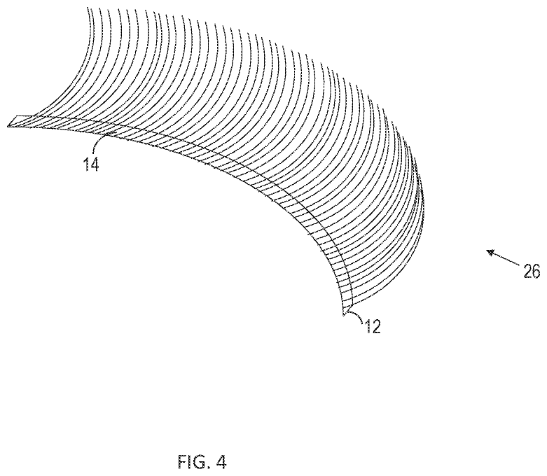

Referring now to FIG. 4, shown therein is a perspective view of the substrate strip 12 having a plurality of artificial hairs 26 attached to the first surface 14. The artificial hairs 26 may have a first end 28 and a second end 30. The first end 28 may be secured, adhered, coupled, or otherwise attached to the first surface 14 of the substrate strip 12. The artificial hairs 26 may be attached to the first surface 14 using an eyelash glue. In one embodiment, the artificial hairs 26 are attached to the first surface 14 using eyelash glue including ethyl 2-cyanoacrylate.

Each of the plurality of artificial hairs 26 may be formed of human hair, animal hair, synthetic materials, or a combination thereof. Each of the artificial hairs 26 may be of any suitable length, and may be of uniform lengths or varying lengths. The plurality of artificial hairs 26 may be secured to the first surface 14 of the substrate strip 12 such that each of the plurality of artificial hairs 26 extends outwardly from the first surface 14. Each of the artificial hairs 26 may be spaced apart from each other when affixed to the first surface 14 of the substrate strip 12. Each of the artificial hairs 26 may extend outwardly in a range of varying degrees, including sharp angles to perpendicular. In one embodiment, the artificial hairs 26 may be arranged in a crisscrossed and/or clumped patterns so as to have an appearance of disorderly looks including wispy and feathered. In another embodiment, the artificial hairs 26 may be arranged in a configuration of alternating patterns of various multi-lengths so as to have an overall irregular and erratic appearance. In another embodiment, one or more of the artificial hairs 26 may be curled or angled at the second end 30. In another embodiment, the artificial hairs 26 may have alternating long-short lengths and may be arranged in a plain, uniform, and orderly configuration such that the artificial hairs 26 extends substantially perpendicular outwardly from the substrate strip 12. Each of the foregoing embodiments are only examples, and as such, the artificial hairs 26 may be arranged in one of more of the identified embodiments, or other arrangements not set forth herein, but would be understood by one in the art.

The plurality of artificial hairs 26 may secured to the first surface 14 through adhesive means, or sewn means, or a combination thereof. In one embodiment, the first end 28 of each of the plurality of artificial hairs 26 may be coated with an adhesive such as, for example, eyelash glue containing ethyl 2-cyanoacrylate, and adhered to the first surface 14 of the substrate strip 12. In another embodiment, the first end 28 of each of the plurality of artificial hairs 26 may be sewn into the first surface 14 of the substrate strip 12 with the fibers 18 forming the substrate strip 12. The plurality of artificial hairs 26 may also be affixed to the first surface 14 by the application of heat. In one embodiment, a machine may also be used to thread and tighten the substrate strip 12 to facilitate the affixation of the plurality of artificial hairs 26 to the substrate strip 12.

The substrate strip 12 with the affixed plurality of artificial hairs 26 may be attached to a wearer's eyelid by engaging the second surface 16 of the substrate strip 12 to a layer of a magnetic formula, such as a magnetic eyeliner, which has been applied to the wearer's eyelid (not shown). The second surface 16 of the substrate strip 12 may be releasably attached to wearer's eyelid, for example, through magnetic means. In one implementation, a wearer may apply a magnetic formula to the wearer's eyelid where the second surface 16 would contact the wearer's eyelid. For example, a wearer may utilize an applicator having a magnetic formula to apply a layer of the magnetic formula to the ridge of the wearer's eyelid and then contact the second surface 16 of the substrate strip 12 to the magnetic formula layer such that the second surface 16 magnetically engages the wearer's eyelid at the magnetic formula layer. In another embodiment, the wearer may apply the magnetic coating layer 24 to the substrate strip 12 and then wearer may utilize an applicator having a magnetic formula to apply a layer of the magnetic formula to the ridge of the wearer's eyelid and then contact the second surface 16 of the substrate strip 12 containing the magnetic coating layer 24 to the magnetic formula layer such that the second surface 16 magnetically engages the wearer's eyelid at the magnetic formula.

### Conclusion

Conventionally, artificial or false eyelashes assemblies are secured to a wearer's eyelid using adhesives or discrete magnetic elements. The use of adhesives has resulted in both injury to a wearer's eyelid and also the false eyelash assembly itself. Similarly, the use of discreet magnetic elements to attach a false eyelash assembly to a wearer's eyelids have also resulted in injury to the wearer and, further, results in the inability of the false eyelash assembly to lay flat on a wearer's eyelid. In accordance with the present disclosure, a false eyelash band and a false eyelash assembly are disclosed to remove the healthy and aesthetic issues plaguing artificial eyelash assemblies employing adhesives and/or discrete magnetic elements. More particularly, a false eyelash band is disclosed having a substrate strip having a first surface configured to secure a plurality of artificial hairs along the length of the substrate strip and an opposing second surface configured to attach the substrate strip to a wearer's eyelid. The substrate strip is formed of a plurality of fibers impregnated with magnetic element particles from a magnetic coating composition including a magnetic substance. The second surface of the substrate strip may also be coated with a magnetic coating layer including a magnetic substance for a stronger hold to the wearer's eyelid.

The foregoing description provides illustration and description, but is not intended to be exhaustive or to limit the inventive concepts to the precise form disclosed. Modifications and variations are possible in light of the above teachings or may be acquired from practice of the methodologies set forth in the present disclosure.

Even though particular combinations of features are recited in the claims and/or disclosed in the specification, these combinations are not intended to limit the disclosure. In fact, many of these features may be combined in ways not specifically recited in the claims and/or disclosed in the specification. Although each dependent claim listed below may directly depend on only one other claim, the disclosure includes each dependent claim in combination with every other claim in the claim set.

What is claimed is:

1. A false eyelash band, comprising:
a substrate strip having a first surface and an opposing second surface, the substrate strip formed by a consolidated plurality of fibers, each of the plurality of fibers impregnated with magnetic element particles from a magnetic coating composition, the first surface configured to secure a plurality of artificial hairs along a length of the substrate strip, the second surface comprising at least a magnetic coating layer comprising a magnetic substance and configured to be attached to a user.

2. The false eyelash band of claim 1, wherein the plurality of fibers are formed of a material including cotton.

3. The false eyelash band of claim 1, wherein the magnetic element particles are black iron oxide particles.

4. The false eyelash band of claim 1, wherein the magnetic coating composition comprises components (i) water, (ii) a solvent, (iii) a magnetic substance (iv) an emulsifier, (v) a film forming agent, (vi) a surfactant, and (vii) a thickener.

5. The false eyelash band of claim 1, wherein the magnetic coating composition comprises components (1) water, (2) propylene glycol, (3) black iron oxide, (4) titanium dioxide, (5) styrene acrylate copolymer, (6) ethylhexylglycerin, and (7) xanthan gum.

6. The false eyelash band of claim 5, wherein the magnetic coating composition further comprises the components (1) water in an amount of about 46 wt %, (2) propylene glycol in an amount of about 6 wt %, (3) black iron oxide in an amount of about 10 wt %, (4) titanium dioxide in an amount of about 23 wt %, (5) styrene acrylate copolymer in an amount of about 14.6 wt %, (6) ethylhexylglycerin in an amount of about 0.3 wt %, and (7) xanthan gum in an amount of about 0.1 wt %.

7. The false eyelash band of claim 1, wherein the magnetic coating layer and the magnetic coating composition are comprised of an identical component.

8. A false eyelash assembly, comprising:
a substrate strip having a first surface and an opposing second surface, the substrate strip formed by a consolidated plurality of fibers, each of the plurality of fibers impregnated with magnetic element particles; and
a magnetic coating layer including a magnetic substance coated on the second surface; and
a plurality of artificial hairs spaced apart and secured along the length of the first surface.

9. The false eyelash assembly of claim 8, wherein the plurality of fibers are formed of a material including cotton.

10. The false eyelash assembly of claim 8, wherein the magnetic coating layer comprises components (i) water, (ii) a solvent, (iii) a magnetic substance, (iv) an emulsifier, (v) a film forming agent, (vi) a surfactant, and (vii) a thickener.

11. The false eyelash assembly of claim 8, wherein the magnetic coating layer comprises components (1) water, (2) propylene glycol, (3) black iron oxide, (4) titanium dioxide, (5) styrene acrylate copolymer, (6) ethylhexylglycerin, (7) xanthan gum.

12. The false eyelash assembly of claim 11, wherein the magnetic coating layer further comprises the components (1) water in an amount of about 46 wt %, (2) propylene glycol in an amount of about 6 wt %, (3) black iron oxide in an amount of about 10 wt %, (4) titanium dioxide in an amount of about 23 wt %, (5) styrene acrylate copolymer in an amount of about 14.6 wt %, (6) ethylhexylglycerin in an amount of about 0.3 wt %, and (7) xanthan gum in an amount of about 0.1 wt %.

14. The false eyelash assembly of claim 8, wherein the magnetic element particles are black iron oxide particles.
